(19) United States
(12) Patent Application Publication
(10) Pub. No.: US 2018/0318181 A1
(43) Pub. Date: Nov. 8, 2018

(54) AGENT AND METHOD FOR HAIR CLEANSING

(71) Applicant: Henkel AG & Co. KGaA, Duesseldorf (DE)

(72) Inventors: Thorsten Knappe, Schenefeld (DE); Tim Bethge, Hamburg (DE)

(73) Assignee: Henkel AG & Co. KGaA, Duesseldorf (DE)

(21) Appl. No.: 15/966,574

(22) Filed: Apr. 30, 2018

(30) Foreign Application Priority Data
May 2, 2017 (DE) 10 2017 207 348.9
Apr. 5, 2018 (DE) 10 2018 205 095.3

(51) Int. Cl. A61K 8/04; A61K 8/19; A61K 8/34; A61K 8/73; A61K 8/89; A61Q 5/02

(52) U.S. Cl. CPC A61K 8/046; A61K 8/19; A61K 8/34; A61K 2800/43; A61K 8/89; A61Q 5/02; A61K 8/732

(57) ABSTRACT

Cosmetic composition containing, relative to its total weight

a) from about 1.0 to about 15 wt. % starch
b) from about 0.05 to about 3.0 wt. % hydrophobized metal oxide powder
c) from about 0.05 to about 3.0 wt. % silicone oil
d) from about 2.0 to about 20 wt. % ethanol
e) from about 40 to about 95 wt. % propellant
and utilization thereof, and method with use of this composition.

### CROSS-REFERENCE TO RELATED APPLICATION

This application claims priority to German Patent Application No. 10 2017 207 348.9, filed May 2, 2017, and also claims priority to German Patent Application No. 10 2018 205 095.3 filed on Apr. 5, 2018, each of which is hereby incorporated in its entirety by reference.

### TECHNICAL FIELD

The application relates to agents and methods for cleansing keratinous fibers, more particularly human hair.

### BACKGROUND

Keratinous fibers are usually cleansed by employing the combined effect of water, soap and mechanical work. For example, hair washing involves first applying a tenside-containing shampoo to damp hair and massaging said shampoo into the hair. If the hair is then rinsed with water, the impurities detached by employing the water, the shampoo and/or the mechanical effect are rinsed out of the hair, the hair thus being cleansed in this manner.

If there is no water available for hair cleansing or the hair cannot be cleansed in the conventional manner for lack of time, hair cleansing by employing a dry shampoo is suitable as an alternative to water-based hair cleansing.

Dry shampoos contain, as an essential active ingredient, a particulate carrier material which, due to its adsorption properties, is able to bind impurities such as fats or sebum present on the hair or scalp. Modified starches are primarily used as carrier materials. When the hair is combed or brushed after the dry shampoo has been applied, the adsorbed impurities are removed from the hair along with the powdery carrier material.

The aerosol spray is a preferred packaging form for dry shampoo. With similar products, the powder suspended in a liquid phase is sprayed onto the hair by employing a propellant. Such agents are described, for example, in international patent applications WO 2013/143792 A2 and WO 2013/143935 A2.

In addition to simple and time-saving hair cleansing, consumers are increasingly making greater demands of modern dry shampoos. These requirements relate to texture and volume, for example. After using the dry shampoo, the hair not only has to appear clean but also coiffed and styled. One solution for this requirement is to use modified silicon oxides (silica). On the one hand, these active ingredients improve the hair texture and volume but the consumer also notices that it feels rough. This effect is one of the major drawbacks when using modified silicon oxides in dry shampoos.

### BRIEF SUMMARY

This disclosure provides a cosmetic composition comprising, relative to its total weight: a) from about 1.0 to about 15 wt. % starch; b) from about 0.05 to about 3.0 wt. % hydrophobized metal oxide powder; c) from about 0.05 to about 3.0 wt. % silicone oil; d) from about 2.0 to about 20 wt. % ethanol; and e) from about 40 to about 95 wt. % propellant. This disclosure also provides a method for cleaning keratinous fibers wherein the cosmetic composition is applied to keratinous fibers.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The present application therefore addresses the problem of producing a dry shampoo with a high adsorption capacity and good application characteristics in the form of an aerosol spray, which promotes texture and volume when used without making the hair feel rough. This problem was solved using a specific combination of active ingredients.

The present application provides:

A cosmetic composition containing, relative to its total weight

a) from about 1.0 to about 15 wt. % starch
b) from about 0.05 to about 3.0 wt. % hydrophobized metal oxide powder
c) from about 0.05 to about 3.0 wt. % silicone oil
d) from about 2.0 to about 20 wt. % ethanol
e) from about 40 to about 95 wt. % propellant.

A Cosmetic composition described above, where the cosmetic composition also contains

f) from about 0 to about 10 wt. % dye(s)
g) from about 0 to about 10 wt. % pigment(s)
with the proviso that the sum of the quantities of ingredients 0 and g) 0.0001 to 10 wt. %, is based on the total weight of the cosmetic composition.

A Cosmetic composition described above, wherein the weight proportion of starches relative to the total weight of the cosmetic composition is from about 1.5 to about 10 wt. % and more particularly from about 2.0 to about 5.0 wt. %.

A Cosmetic composition described above, where the starch used is rice starch.

A Cosmetic composition described above, wherein the percentage weight of the hydrophobized metal oxide powder to the total weight of the composition is from about 0.07 to about 2.0 wt. %, particularly from about 0.1 to about 1.0 wt. %.

A Cosmetic composition described above, wherein a hydrophobized silicate obtained by silanizing pyrogenic silica is used as hydrophobized metal oxide powder.

A Cosmetic composition described above, wherein the percentage weight of the silicon oil to the total weight of the composition is from about 0.07 to about 2.0 wt. %, particularly from about 0.1 to about 1.0 wt. %.

A Cosmetic composition described above, wherein particular preference is given to a silicon oil selected from the group of dialkyl and alkylarylsiloxanes from the group polydimethylsiloxane, cyclopentasiloxane, cyclohexasiloxane, and methylphenylpolysiloxane, and particularly from the group of polydimethylsiloxanes.

A Cosmetic composition described above, wherein the weight ratio of hydrophobized metal oxide powder to silicon oil is from about 5:1 to about 1:5, preferably from about 3:1 to about 1:3 and particularly from about 2:1 to about 1:2.

A Cosmetic composition described above, wherein the percentage by weight of ethanol of the total weight of the cosmetic composition is from about 4.0 to about 15 wt. %, preferably from about 7.0 to about 12 wt. %.

A Cosmetic composition described above, wherein the percentage by weight of propellant of the total weight of the composition is from about 60 to about 92 wt. %, preferably from about 80 to about 90 wt. %.

A Cosmetic composition described above, wherein a propellant from the group of propane, propane/butane mixtures and dimethyl ether, more particularly from the group of propane/butane mixtures is used.

A Cosmetic composition described above, where the cosmetic composition also contains cationic surfactant.

A Cosmetic composition described above, where the cosmetic composition also contains cationic surfactant from the group of quaternary ammonium compounds, preferably cationic surfactant from the group cetyl trimethyl ammonium chloride, stearyltrimethylammoniumchloride, distearyldimethylammoniumchloride lauryldimethylammonium chloride, benzyl ammonium chloride and lauryl dimethyl ammonium chloride and tricetylmethylammoniumchloride, particularly preferred cetyltrimethylammoniumchloride.

A Cosmetic composition described above, where the cosmetic composition contains less than about 1.0 wt. % water, preferably less than about 0.5 wt. % and, particularly, less than about 0.1 wt. %.

A Cosmetic composition described above, containing, in terms of the total weight—from about 0.0001 to about 10 wt. %, preferably from about 0.01 to about 9.5 wt. %, preferably from about 0.05 to about 9 wt. %, more preferably from about 0.1 to about 8.5 wt. % and, in particular, from about 0.25 to about 8 wt. % dye(s).

A Cosmetic composition described above, containing, in terms of the total weight—from about 0.0001 to about 10 wt. %, preferably from about 0.01 to about 9.5 wt. %, preferably from about 0.05 to about 9 wt. %, more preferably from about 0.1 to about 8.5 wt. % and, in particular, from about 0.25 to about 8 wt. % pigment(s).

A Cosmetic composition described above, containing ingredients 0 and/or (g) in the form of a starch-dye compound and/or a starch-pigment compound, preferably in the form of colored and/or pigmented rice starch.

A Cosmetic composition described above, wherein the cosmetic composition contains at least about 90 wt. %, preferably at least about 94 wt. % and particularly at least about 98 wt. % of the components

a) starch
b) hydrophobized metal oxide powder
c) silicone oil
d) ethanol
e) propellant.

Cosmetic agent, comprising

a) a cosmetic composition described above; and
b) an aerosol dispensing container.

Use of a cosmetic composition described above cleansing keratinous fibers, more particularly human hair.

Use of a cosmetic composition described above for temporary deformation of keratinous fibers, more particularly human hair.

A Method for cleansing keratinous fibers, more particularly human hair, wherein the cosmetic composition described above is applied to keratinous fibers.

A Method described above, wherein the cosmetic composition is applied to the hair, left for a period of from about 1 to about 10 minutes and then removed from the hair.

The compositions exist preferably in the form of a dispersion of solid particles in a liquid phase from ethanol and propellant.

A first essential component of the compositions is the starch.

Starch is a reserve carbohydrate, which is stored by many plants in the form of, usually, from about 1 to about 200 μm starch grains (granulate) in various plant parts, e.g. bulbs or roots, grain seeds, fruits, and also in the core. Starch belongs to the family of homoglycans and a poly-condensation product of D-glucose. Starch includes three structurally different polymers of d-glucopyranose, specifically amylose, amylopectin and a so-called intermediate fraction.

A preferred starch compound usable is selected from at least one—possibly modified—poly-condensation product of D-glucose, obtained from the starch of potatoes, corn, rice, peas, acorns, chestnuts, barley, wheat, bananas, sago, sorghum, oats, rye, beans, batata, maranta or manioc. More preferably, the cosmetic composition contains at least one starch compound, which is tapioca starch, potato starch, corn starch or rice starch. As contemplated herein, the agent likewise comprises mixtures of the aforementioned starch compounds.

The use of rice starch is most preferred. Starch compounds based on rice starch are, for example, under the designation Remy DR KA (INCI designation: Oryza Sativa (Rice) Starch, CAS number 9005-25-8) from the company Bene 0 Remy Industries or under the designation Rice Starch D.S.A. 7 (INCI designation: Oryza Sativa (Rice) Starch, Cetrimonium Chloride; CAS number 9005-25-8) available from Agrana.

The cosmetic effect and applicability of the cosmetic can be beneficially influenced by the starch content. Preferred cosmetic compositions are exemplified by the fact that this composition contains in terms of their total weight from about 1.5 to about 10 wt. % and, in particular, from about 2.0 to about 5.0 wt. % of starch.

The cosmetic compositions contain the hydrophobized metal oxide powder as a second essential constituent.

As contemplated herein, hydrophobically modified or hydrophobized means metal oxides that were modified at least on the surface of the particle in such a manner that the modified particles are moistened by water to a lesser degree than the non-modified particles. Silanized, hydrophobized silica are metal oxides are more particularly preferred. A preferred reagent for silanizing the metal oxide is at least one representative from the group formed from silanes, halogen silanes, alkoxysilanes and silazanes. Preferred suitable hydrophobized metal oxides of the hydrophobized metal oxide powder are selected from at least one representative of the group formed from hydrophobized silicates, hydrophobized aluminum silicates, hydrophobized titanium dioxide and hydrophobized silica. Hydrophobized silicates have proved to be particularly suitable for producing the cosmetic compositions, with pyrogenic silica post-treated by silylation or by reacting with polydimethylsiloxane offering particular advantages.

The cosmetic compositions preferably contain hydrophobized silicon dioxide. The type of hydrophobic modified silicon dioxide is not limited in principle, however, due to the cosmetic properties of corresponding compositions such as water-repellent metal oxide powder, at least one silanized, hydrophobized silicon dioxide preferably used.

A preferred reagent for silanizing the silica is at least one representative from the group formed from silanes, halogen silanes, alkoxysilanes and silazanes.

Preferred representatives of the group of silanes are hexa(C₁-C₂₀)alkyldisilane, more particularly hexamethyldisilane.

Where a halogen silane is used as the silylating agent, the preferred halogen silane is at least one compound from the group formed from the following compounds

[(C₁-C₂₀)Alkyl]_{z′}SiX_{(4-z′)}
X₃Si[(CH₂)ₙ—R]
X₂[(C₁-C₂₀)Alkyl]Si(CH₂)ₙ—R
[(C₁-C₂₀)Alkyl]_{(y′+1)}[R—(CH₂)ₙ]_{(2-y′)}SiX
wherein
X means a chlorine, bromine or iodine atom,
z′ is a numeral 1, 2 or 3,
y′ is a numeral 0, 1 or 2
n is an integer from 1 to 20 and
R denotes a radical from
(C₁-C₁₀)Alkyl-, Aryl-, (C₁-C₆)Perfluoroalkyl-, —NH₂, —N₃, —SCN, —CH═CH₂, —O(O)C—C(CH₃)═CH₂, —OCH₂—CH═CH₂,

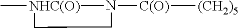

—NH—C(O)O-Me, —NH—C(O)O-Et, —NH—(CH₂)₃—Si(O(C₁-C₆)alkyl)₃.
Where an alkoxysilane is used as the silylating agent, the preferred alkoxysilane is at least one compound from the group formed from the following compounds
[(C₁-C₂₀)AlkylO]_{z}Si(C₁-C₂₀)Alkyl_{(4-z)}
[(C₁-C₂₀)AlkylO]_{z}Si[(CH₂)ₙ—R]_{(4-z)}
[(C₁-C₂₀)AlkylO]₂[(C₁-C₂₀)Alkyl]Si(CH₂)ₙ—R
[(C₁-C₂₀)AlkylO][(C₁-C₂₀)Alkyl]₂Si(CH₂)ₙ—R
[(C₁-C₂₀)AlkylO][(C₁-C₂₀)Alkyl]Si[(CH₂)ₙ—R]₂
(C₁-C₂₀Alkyl)₃SiO—C(CH₃)═N—Si(C₁-C₂₀)Alkyl₃,
wherein
n is an integer from about 1 to about 20 and
z means a numeral 1, 2, or 3
R denotes a radical from
(C₁-C₂₀)Alkyl-, Aryl-, (C₁-C₆)Perfluoroalkyl-, —NH₂, —N₃, —SCN, —CH═CH₂, —O(O)C—C(CH₃)═CH₂, —OCH₂—CH═CH₂,

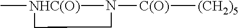

—NH—C(O)O-Me, —NH—C(O)O-Et, —NH—(CH₂)₃—Si(O(C₁-C₆)alkyl)₃.
A preferred silazane is at least one compound selected from the disilazane class, more preferably at least one disilazane compound of the formula

R′₂R″Si—NH—SiR′₂R″

wherein
R′ means a (C₁-C₂₀)Alkyl group and
R″ means a (C₁-C₂₀)Alkyl group or a vinyl group. A more preferred silazane is
hexamethyldisilazane.

All of the aforementioned alkyl groups, whether (C₁-C₆)Alkyl, (C₁-C₁₀)Alkyl or (C₁-C₂₀)-Alkyl, can be both cyclical and also linear and/or branched. Examples of alkyl groups suitable for use are methyl, ethyl, n-Propyl, isopropyl, n-Butyl, cyclopentyl, cyclohexyl, n-Decyl, lauryl, myristyl, cetyl, stearyl, isostearyl and behenyl.

An example of an aryl group is a phenyl group.

Examples of a (C₁-C₆)Perfluoroalkyl group are trifluoromethyl, perfluoroethyl, perfluoropropyl and perfluorohexyl.

The preference is for hydrophobized silica obtained by silanizing pyrogenic silica.

Silanized, hydrophobized silica are preferably selected from at least one compound of the group formed from trimethylsilylate-coated silica, dimethylsilylate-coated silica, octylsilylate-coated silica.

Preferred cosmetic compositions are exemplified by the fact that the modified hydrophobic metal oxide powder is selected from the group of hydrophobized silicates obtained by silanizing pyrogenic silica.

Numerous suitable hydrophobically modified silicas are commercially available. Examples are Aerosil® R104 V, Aerosil® R106, Aerosil® R202, Aerosil® R805, Aerosil® R812, Aerosil® R812S, Aerosil® R972 and Aerosil® R8200, all from Degussa, as well as HDK® H2000, HDK® H2050 and HDK® H3004, all from Wacker. Use of the silica with the INCI designation Silica Dimethyl Silylate, which is sold by Degussa under the trade name of Aerosil® R972 is most preferred. In summary, preference is given to cosmetic compositions in which the hydrophobically modified metal oxide powder is selected from compounds with INCI designations Silica Dimethyl Silylate (e.g. Aerosil R792, Aerosil R794), Silica Dimethicone Silylate (e.g. Aerosil R202) and Silica Silylate (e.g. Aerosil R805, Aerosil R812, Aerosil R816). Particularly preferred are compounds with the INCI designation Silica Dimethyl Silylate.

Hydrophobically modified silicates with the INCI designation Silica Dimethyl Silylate improve the applicability and cosmetic effect of the cosmetic compositions. The manufacture of particularly preferred hydrophobized silicon dioxide with the INCI designation Silica Dimethyl Silylate can take place, for example, by reacting pyrogenic silica with dimethyldichlorosilane. A particularly preferred subject-of the present application is therefore a cosmetic composition that contains a hydrophobically modified metal oxide powder, obtained by reacting pyrogenic silica with dimethyldichlorosilane.

The particle diameter of primary particles of the preferred hydrophobically modified metal oxides, in particular, hydrophobically modified metal oxides with the INCI designation Silica Dimethyl Silylate is preferably less than about 5 μm, more preferably less than about 1 μm, and, in particular, between from about 1 and about 50 Nm.

Also preferred are such hydrophobically modified metal oxides, in particular hydrophobically modified metal oxides with the INCI designation Silica Dimethyl Silylate, offering a specific surface area according to BET of between from about 10 and about 400 m²/g, preferably between from about 40 to about 300 m²/g and, in particular, from about 80 to about 150 m²/g.

Cosmetic composition according to one of the points above, wherein the percentage weight of the hydrophobized metal oxide powder to the total weight of the composition is from about 0.07 to about 2.0 wt. %, particularly from about 0.1 to about 1.0 wt. %.

The third essential constituent of the cosmetic compositions is silicone oil. Particular preference is given to the use of silicone oil from the group of dialkyl and alkylarylsiloxanes, more preferably from the group polydimethylsiloxane, cyclopentasiloxane, cyclohexasiloxane, and methylphenylpolysiloxane, and most preferred from the group of polydimethylsiloxanes.

For the cosmetic effect but also for the packaging and application it has proved to be beneficial if the percentage by weight of the silicone oil of the total weight of the cosmetic composition is from about 0.07 to about 2.0 wt. % and, in particular, from about 0.1 to about 1.0 wt. %.

In addition to the absolute content of hydrophobized metal oxide powder and silicone oil, the weight ratio of these two active ingredients has also proved to be relevant for the cosmetic effect and applicability. Cosmetic composition in which the weight ratio of hydrophobized metal oxide powder to silicone oil is from about 5:1 to about 1:5, preferably from about 3:1 to about 1:3 and from about 2:1 to about 1:2, are preferred due to their advantageous cosmetic and technical characteristics.

The fourth essential component of the cosmetic compositions is ethanol, whose percentage by weight of the total weight of composition is preferably from about 4.0 to about 15 wt. % and in particular from about 7.0 to about 12 wt. %.

The cosmetic compositions are exemplified by low water content. The percentage by weight of water of the total weight of these compositions is preferably less than about 1.0 wt. %, especially preferably less than about 0.5 wt. % and, in particular, less than about 0.1 wt. %.

A final essential component of the cosmetic compositions is the propellant. It has proved to be particularly cosmetically and technically advantageous if the propellant has a proportion by weight of the total weight of the composition of from about 60 to about 92 wt. %, and in particular from about 80 to about 90 wt. %.

Preferred propellants (propellant gases) are selected from propane, propene, n-butane, isobutane, isobutene, n-pentane, pentene, isopentane, isopentene, methane, ethane, dimethyl ether, nitrogen, air, oxygen, nitrous oxide, dichlorofluoromethane, chlorodifluoromethane, chlorofluoromethane, 1,1,2,2-tetrachloro-1-fluoroethane, 1,1,1,2-tetrachloro-2-fluoroethane, 1,2,2-trichloro-1,1-difluoroethane, 1,1,2-trichloro-1,2-difluoroethane, 1,1,1-trichloro-2,2-difluoroethane, 2,2-dichloro-1,1,1-trifluoroethane, 1,2-dichloro-1,1,2-trifluoroethane, 2-chloro-1,1,1,2-tetrafluoroethane, 1-chloro-1,1,2,2-tetrafluoroethane, 1,1,2-trichloro-2-fluoroethane, 1,2-dichloro-1,2-difluoroethane, 1,2-dichloro-1,1-difluoroethane, 1-chloro-1,2,2-trifluoroethane, 2-chloro-1,1,1-trifluoroethane, 1-chloro-1,1,2-trifluoroethane, 1,2-dichloro-1-fluoroethane, 1,1-dichloro-1-fluoroethane, 2-chloro-1,1-difluoroethane, 1-chloro-1,1-difluoroethane, 1-chloro-2-fluoroethane, 1-chloro-1-fluoroethane, 2-chloro-1,1-difluoroethene, 1,1,1,3-tetrafluoroethane, heptafluoro-n-propane, perfluoroethane, monochlorodifluoromethane, 1,1-difluoroethane, both individually and in combination.

The use of propane, propane/butane mixtures or dimethyl ether, most preferably the use of propane/butane mixtures, is preferred.

For the manufacture and application of the cosmetic compositions, it has proved to be beneficial if they include for the most part of the previously described essential ingredients. Cosmetic compositions including of at least about 90 wt. %, preferably at least about 94 wt. % and, in particular, at least about 98 wt. % of the components

a) starch
b) hydrophobized metal oxide powder
c) silicone oil
d) ethanol
e) propellant
are preferred for this reason.

The composition of some other preferred cosmetic compositions can be found in the table below (values in wt. % relative to the total weight of the cosmetic composition and/or relative to the weight of the starch particles).

| | Formula | Formula | Formula | Formula | Formula |
|---|---|---|---|---|---|
| | 1 | 2 | 3 | 4 | 5 |
| Starch | 1.0 to 15 | 1.5 to 10 | 1.5 to 10 | 2.0 to 5.0 | 2.0 to 5.0 |
| hydrophobized metal | 0.05 to 3.0 | 0.07 to 2.0 | 0.07 to 2.0 | 0.1 to 1.0 | 0.1 to 1.0 |
| oxide powder | | | | | |
| Silicon oil | 0.05 to 3.0 | 0.07 to 2.0 | 0.07 to 2.0 | 0.1 to 1.0 | 0.1 to 1.0 |
| Ethanol | 2.0 to 20 | 4.0 to 15 | 4.0 to 15 | 4.0 to 15 | 7.0 to 12 |
| Propellant | 40 to 95 | 60 to 92 | 60 to 92 | 60 to 92 | 80 to 90 |
| Optional additives | ad 100 | ad 100 | ad 100 | ad 100 | ad 100 |
| | Formula | Formula | Formula | Formula | Formula |
| | 6 | 7 | 8 | 9 | 10 |
| Rice starch | 1.0 to 15 | 1.5 to 10 | 1.5 to 10 | 2.0 to 5.0 | 2.0 to 5.0 |
| hydrophobized metal | 0.05 to 3.0 | 0.07 to 2.0 | 0.07 to 2.0 | 0.1 to 1.0 | 0.1 to 1.0 |
| oxide powder | | | | | |
| Silicon oil | 0.05 to 3.0 | 0.07 to 2.0 | 0.07 to 2.0 | 0.1 to 1.0 | 0.1 to 1.0 |
| Ethanol | 2.0 to 20 | 4.0 to 15 | 4.0 to 15 | 4.0 to 15 | 7.0 to 12 |
| Propellant | 40 to 95 | 60 to 92 | 60 to 92 | 60 to 92 | 80 to 90 |
| Optional additives | ad 100 | ad 100 | ad 100 | ad 100 | ad 100 |
| | Formula | Formula | Formula | Formula | Formula |
| | 11 | 12 | 13 | 14 | 15 |
| Starch | 1.0 to 15 | 1.5 to 10 | 1.5 to 10 | 2.0 to 5.0 | 2.0 to 5.0 |
| hydrophobized silica | 0.05 to 3.0 | 0.07 to 2.0 | 0.07 to 2.0 | 0.1 to 1.0 | 0.1 to 1.0 |
| Silicon oil | 0.05 to 3.0 | 0.07 to 2.0 | 0.07 to 2.0 | 0.1 to 1.0 | 0.1 to 1.0 |
| Ethanol | 2.0 to 20 | 4.0 to 15 | 4.0 to 15 | 4.0 to 15 | 7.0 to 12 |
| Propellant | 40 to 95 | 60 to 92 | 60 to 92 | 60 to 92 | 80 to 90 |
| Optional additives | ad 100 | ad 100 | ad 100 | ad 100 | ad 100 |
| | Formula | Formula | Formula | Formula | Formula |
| | 16 | 17 | 18 | 19 | 20 |
| Starch | 1.0 to 15 | 1.5 to 10 | 1.5 to 10 | 2.0 to 5.0 | 2.0 to 5.0 |
| Silica dimethyl silylate | 0.05 to 3.0 | 0.07 to 2.0 | 0.07 to 2.0 | 0.1 to 1.0 | 0.1 to 1.0 |
| Silicon oil | 0.05 to 3.0 | 0.07 to 2.0 | 0.07 to 2.0 | 0.1 to 1.0 | 0.1 to 1.0 |
| Ethanol | 2.0 to 20 | 4.0 to 15 | 4.0 to 15 | 4.0 to 15 | 7.0 to 12 |
| Propellant | 40 to 95 | 60 to 92 | 60 to 92 | 60 to 92 | 80 to 90 |
| Optional additives | ad 100 | ad 100 | ad 100 | ad 100 | ad 100 |
| | Formula | Formula | Formula | Formula | Formula |
| | 21 | 22 | 23 | 24 | 25 |
| Starch | 1.0 to 15 | 1.5 to 10 | 1.5 to 10 | 2.0 to 5.0 | 2.0 to 5.0 |
| hydrophobized metal | 0.05 to 3.0 | 0.07 to 2.0 | 0.07 to 2.0 | 0.1 to 1.0 | 0.1 to 1.0 |
| oxide powder | | | | | |
| Polydimethylsiloxane | 0.05 to 3.0 | 0.07 to 2.0 | 0.07 to 2.0 | 0.1 to 1.0 | 0.1 to 1.0 |
| Ethanol | 2.0 to 20 | 4.0 to 15 | 4.0 to 15 | 4.0 to 15 | 7.0 to 12 |
| Propellant | 40 to 95 | 60 to 92 | 60 to 92 | 60 to 92 | 80 to 90 |
| Optional additives | ad 100 | ad 100 | ad 100 | ad 100 | ad 100 |
| | Formula | Formula | Formula | Formula | Formula |
| | 26 | 27 | 28 | 29 | 30 |
| Starch | 1.0 to 15 | 1.5 to 10 | 1.5 to 10 | 2.0 to 5.0 | 2.0 to 5.0 |
| hydrophobized metal | 0.05 to 3.0 | 0.07 to 2.0 | 0.07 to 2.0 | 0.1 to 1.0 | 0.1 to 1.0 |
| oxide powder | | | | | |
| Silicon oil | 0.05 to 3.0 | 0.07 to 2.0 | 0.07 to 2.0 | 0.1 to 1.0 | 0.1 to 1.0 |
| Ethanol | 2.0 to 20 | 4.0 to 15 | 4.0 to 15 | 4.0 to 15 | 7.0 to 12 |
| Propane-butane | 40 to 95 | 60 to 92 | 60 to 92 | 60 to 92 | 80 to 90 |
| Optional additives | ad 100 | ad 100 | ad 100 | ad 100 | ad 100 |
| | Formula | Formula | Formula | Formula | Formula |
| | 31 | 32 | 33 | 34 | 35 |
| Rice starch | 1.0 to 15 | 1.5 to 10 | 1.5 to 10 | 2.0 to 5.0 | 2.0 to 5.0 |
| hydrophobized silica | 0.05 to 3.0 | 0.07 to 2.0 | 0.07 to 2.0 | 0.1 to 1.0 | 0.1 to 1.0 |
| Silicon oil | 0.05 to 3.0 | 0.07 to 2.0 | 0.07 to 2.0 | 0.1 to 1.0 | 0.1 to 1.0 |
| Ethanol | 2.0 to 20 | 4.0 to 15 | 4.0 to 15 | 4.0 to 15 | 7.0 to 12 |
| Propellant | 40 to 95 | 60 to 92 | 60 to 92 | 60 to 92 | 80 to 90 |
| Optional additives | ad 100 | ad 100 | ad 100 | ad 100 | ad 100 |
| | Formula | Formula | Formula | Formula | Formula |
| | 36 | 37 | 38 | 39 | 40 |
| Rice starch | 1.0 to 15 | 1.5 to 10 | 1.5 to 10 | 2.0 to 5.0 | 2.0 to 5.0 |
| Silica dimethyl silylate | 0.05 to 3.0 | 0.07 to 2.0 | 0.07 to 2.0 | 0.1 to 1.0 | 0.1 to 1.0 |
| Silicon oil | 0.05 to 3.0 | 0.07 to 2.0 | 0.07 to 2.0 | 0.1 to 1.0 | 0.1 to 1.0 |
| Ethanol | 2.0 to 20 | 4.0 to 15 | 4.0 to 15 | 4.0 to 15 | 7.0 to 12 |
| Propellant | 40 to 95 | 60 to 92 | 60 to 92 | 60 to 92 | 80 to 90 |
| Optional additives | ad 100 | ad 100 | ad 100 | ad 100 | ad 100 |
| | Formula | Formula | Formula | Formula | Formula |
| | 41 | 42 | 43 | 44 | 45 |
| Rice starch | 1.0 to 15 | 1.5 to 10 | 1.5 to 10 | 2.0 to 5.0 | 2.0 to 5.0 |
| hydrophobized silica | 0.05 to 3.0 | 0.07 to 2.0 | 0.07 to 2.0 | 0.1 to 1.0 | 0.1 to 1.0 |
| Polydimethylsiloxane | 0.05 to 3.0 | 0.07 to 2.0 | 0.07 to 2.0 | 0.1 to 1.0 | 0.1 to 1.0 |
| Ethanol | 2.0 to 20 | 4.0 to 15 | 4.0 to 15 | 4.0 to 15 | 7.0 to 12 |
| Propellant | 40 to 95 | 60 to 92 | 60 to 92 | 60 to 92 | 80 to 90 |
| Optional additives | ad 100 | ad 100 | ad 100 | ad 100 | ad 100 |
| | Formula | Formula | Formula | Formula | Formula |
| | 46 | 47 | 48 | 49 | 50 |
| Rice starch | 1.0 to 15 | 1.5 to 10 | 1.5 to 10 | 2.0 to 5.0 | 2.0 to 5.0 |
| Silica dimethyl silylate | 0.05 to 3.0 | 0.07 to 2.0 | 0.07 to 2.0 | 0.1 to 1.0 | 0.1 to 1.0 |
| Polydimethylsiloxane | 0.05 to 3.0 | 0.07 to 2.0 | 0.07 to 2.0 | 0.1 to 1.0 | 0.1 to 1.0 |
| Ethanol | 2.0 to 20 | 4.0 to 15 | 4.0 to 15 | 4.0 to 15 | 7.0 to 12 |
| Propellant | 40 to 95 | 60 to 92 | 60 to 92 | 60 to 92 | 80 to 90 |
| Optional additives | ad 100 | ad 100 | ad 100 | ad 100 | ad 100 |

Particularly preferred cosmetic compositions also contain

f) from about 0 to about 10 wt. % dye(s)
g) from about 0 to about 10 wt. % pigment(s)
with the proviso that the sum of the quantities of ingredients f) and g) from about 0.0001 to about 10 wt. %, is based on the total weight of the cosmetic composition.

In other words, the compositions can contain about 0 wt. % pigments (then there must be at least one pigment in an amount of at least about 0.0001 wt. %), however, the dye content but can also be about 10 wt. % (then there can be no pigment. Regardless of whether

only one dye,
several dyes,
only one pigment
several pigments,
one dye and several pigments
several dyes and one pigment
several dyes and several pigments
are used, the total amount of all dyes and pigments (also referred to as a total amount of coloring components) in the cosmetic composition is from about 0.0001 to about 10 wt. %, relative to the total weight of the cosmetic composition.

It has been shown that such cosmetic compositions, colored and/or using dyes or pigments, not only increase the hair volume and increase the volume and the feeling of having “more hair”, but coloring the products at the same time can make it possible to cover bald patches, without this rubbing off on hands or clothing and pillows.

Preferred cosmetic compositions are exemplified by the fact that they contain dye(s) in from about 0.0001 to about 10 wt. %, preferably from about 0.01 to about 9.5 wt. %, preferably from about 0.05 to about 9 wt. %, more preferably from about 0.1 to about 8.5 wt. % and, in particular, from about 0.25 to about 8 wt. %, calculated based on the total weight of the cosmetic composition.

Dyes are colorants—chromophoric substances which, in contrast to pigments, are soluble in the application medium (such as water, oil or other solvents).

Depending on the desired color, color intensity and authenticity properties of the resulting coloring, a dye or a mixture of dyes can be selected. In this case, all the usual dyes in the field of cosmetics and commercially available dyes can be used in the context of the present disclosure.

Preferred cosmetic compositions are exemplified by the fact that they contain pigment(s) in from about 0.0001 to about 10 wt. %, preferably from about 0.01 to about 9.5 wt. %, preferably from about 0.05 to about 9 wt. %, more preferably from about 0.1 to about 8.5 wt. % and, in particular, from about 0.25 to about 8 wt. %, calculated based on the total weight of the cosmetic composition.

Basically, all types of pigments that are insoluble in water, for example, natural inorganic pigments (also referred to as mineral pigments) are suitable. These pigments mainly contain sulphides and oxides. Examples of these pigments are Ocker (Fe(OOH); Pigment Yellow 43), burned Siena (Fe₂O₃; Pigment Red 102), Umbra (Fe₂O₃ x MnO₂; Pigment Brown 7:x), cinnabar (β-HgS, PR 106), lapis lazuli (Ultramarine, Na₆Al₆Si₆O₂₄ x Na₂Sn; Pigment Blue 29), Azurit (basic copper carbonate, Cu₃[OH/CO₃]₂; PB 30), green earth (FeO-containing silicate; Pigment Green 23), malachite (Cu₂[(OH)₂, CO₃]) and carbon black (carbon (graphite), Pigment Black 9). In relation to the avoidance of unwanted visible residues or blooms or water-resistant temporary coloring of the fibers, however, the use of synthetic inorganic pigments have proven beneficial. Synthetic inorganic pigments are produced by chemical and/or physical conversion (digestion, precipitation, annealing), for example. Including in particular: white pigments (titanium dioxide (TiO₂), Pigment White PW 6; zinc sulfide (ZnS), PW 7; zinc oxide (ZnO), PW 4; antimony white (Sb₂O₃), PW 11; lithopones (ZnS/BaSO₄), PW 5; lead white (2PbCO₃ x Pb(OH)₂), PW 1), subordinate white fillers (calcium carbonate, PW 18; talcum, PW 26 and barium sulfate, PW 21); black pigments (manganese black, spinel black and pigment soots (graphite hydrocarbon); gloss pigments (absorption pigments, metal pigments or metal effect pigments and pearlescent pigments), as well as inorganic color pigments (iron oxide pigments, iron blue pigments, ultramarine pigments, as well as the bleach chromate pigments, chromium oxide pigments, cadmium pigments and bismuth vanadate pigments, which are less suitable due to their toxicological properties).

Preferred synthetic inorganic pigments are metallic pigments or metal effect pigments made from powdered metals or metal alloys, such as aluminum bronzes (metal: Al), gold bronzes (metal: Cu, Cu—Al— or Cu—Zn-allow), silver bronzes (metal:) Cu—Zn—Ni), fire colored bronzes (metal: oxidized Cu—Zn) as well as patent bronzes (metal: Cu—Zn—(Ni)+dye).

Further preferred synthetic inorganic pigments are pearlescent pigments, including multiple layers of different refractive index. Examples of such pearlescent pigments are magnesium stearate, zinc stearate and lithium stearate or ethyleneglycol distearate or polyethylene terephthalate and pearlescent pigments comprising essentially glimmer, titanium dioxide (titanium dioxide glimmer), bismuth oxychloride or guanine, and which can also be covered with colored oxide layers (e.g. iron oxide or chromium oxide). Glimmer-based and glimmer/metal oxide-based pearlescent pigments are particularly preferred pearlescent pigments. Glimmers are layer silicates. The most important representatives of such silicates as muscovite, phlogopite, paragonite, biotite, lepidolite and margarite. To produce the pearlescent pigments in conjunction with metal oxides, the glimmer, primarily muscovite or phlogopite, is coated with a metal oxide. Suitable metal oxides include TiO₂, Cr₂O₃ and Fe₂O₃. Interference pigments and pearlescent pigments are obtained, as preferred pearlescent pigments, by coating. Said pearlescent pigment types have color effects, in addition to a glittering optical effect. Moreover, the usable pearlescent pigments can additionally contain a color pigment, which is not derived from a metal oxide.

Most preferred pearlescent pigments are pigments which are marketed by Merck under the trade name Colorona®, wherein the Colorona® pigments contain red-brown (from about 47-57 wt. % Muscovit Mica (KH₂(AlSiO₄)₃), from about 43-50 wt. % Fe₂O₃ (INCI: Iron Oxides CI 77491), <3 wt. % TiO₂ (INCI: Titanium Dioxide CI 77891), Colorona® Blackstar Blue (from about 39-47 wt. % Muscovit Mica (KH₂(AlSiO₄)₃), from about 53-61 wt. % Fe₃O₄ (INCI: Iron Oxides CI 77499)), Colorona® Siena Fine (from about 35-45 wt. % Muscovit Mica (KH₂(AlSiO₄)₃), from about 55-65 wt. % Fe₂O₃ (INCI: Iron Oxides CI 77491)), Colorona® Aborigine Amber (from about 50-62 wt. % Muscovit Mica (KH₂(AlSiO₄)₃), from about 36-44 wt. % Fe3O4 (INCI: Iron Oxides CI 77499), from about 2-6 wt. % TiO₂ (INCI: Titanium Dioxide CI 77891)), Colorona® Patagonian Purple (from about 42-54 wt. % Muscovit Mica (KH₂(AlSiO₄)₃), from about 26-32 wt. % Fe₂O₃ (INCI: Iron Oxides CI 77491), from about 18-22 wt. % TiO₂ (INCI: Titanium Dioxide CI 77891), from about 2-4 wt. % Prussian blue (INCI: Ferric Ferrocyanide CI 77510)), Colorona® Chameleon (from about 40-50 wt. % Muscovit Mica (KH₂(AlSiO₄)₃), from about 50-60 wt. % Fe₂O₃ (INCI: Iron Oxides CI 77491)) and Silk® Mica (>98 wt. % Muscovit Mica (KH₂(AlSiO₄)₃)) are most preferred.

A group of particularly preferred pigments is formed from the chromophoric synthetic iron oxides. Particularly preferred representatives from this substance class are Pigment Brown 6 (CI No 77491), Pigment Red 101 (CI No 77491), Pigment Yellow 42 (CI No 77492), Pigment Black 11 (CI No 77499), as well as mixtures of said pigments.

Particularly preferred usable pigments are exemplified by good sprayability in the compositions as contemplated herein, a uniform spray pattern, as well as extremely low tendency to clog the spray nozzles. Here, extremely preferred cosmetic compositions are exemplified by the fact that they contain at least one pigment from the group CI12490, CI14700, CI14720, CI15510, CI15985, CI45380, CI47005, CI60730, CI61565, CI73360, CI74160, CI77007, CI77019, CI77288, CI77289, CI77491, CI77492, where the total amount of pigment(s) from this group is from about 0.0001 to about 10 wt. %, preferably from about 0.01 to about 9.5 wt-%, preferably from about 0.05 to about 9 wt. %, more preferably from about 0.1 to about 8.5 wt. % and, in particular, from about 0.25 to about 8 wt. %.

Using native and/or physically modified rice starches in combination with dyes and/or pigments achieves a particularly high level of adhesion of the dyes and/or pigments to the starch. This means that, in addition to outstanding cleaning performance, long-lasting temporary hair coloring is also possible.

As a result, it is possible and preferable to use pre-colored and/or pre-pigmented starches that can be sprayed with the cosmetic compositions and are extremely effective. Particularly preferred cosmetic compositions are therefore exemplified by the fact that they contain ingredient f) and/or (g) in the form of a starch-dye compound and/or a starch-pigment compound, preferably in the form of colored and/or pigmented rice starch.

In addition, it has been found that the composition of the starch particles has proved to be relevant for the cosmetic effect. Therefore, the preferred particles used have a specific proportion of the native and/or physically modified starch, in particular, rice starch. It is therefore preferable when the starch-dye-compound and/or starch-pigment compound contains at least one starch, in particular a physically modified rice starch, in a total amount of from about 70 to about 96 wt. %, in particular from about 80 to about 94 wt. %, calculated based on the total weight of the particle (compound). The use of particles, which have a high weight of physically modified rice starch, leads to a particularly homogeneous spray pattern and preferred adhesion of color to the keratin fibers.

In addition to the essential ingredients described above, the cosmetic compositions can contain further optional active substances or excipients.

The first group of optional ingredients are cationic surfactants. Particularly preferred is the use of cationic surfactants from the group of quaternary ammonium compounds, preferably from the group cetyl trimethyl ammonium chloride, stearyltrimethylammoniumchloride, distearyldimethylammoniumchloride, lauryldimethylammonium chloride, benzyl ammonium chloride and lauryl dimethylbenzyl ammoniumchloride, most preferably cetyltrimethylammoniumchloride.

Nourishing agents are also suitable active ingredients.

As the nourishing agent, the composition can contain at least one protein hydrolysate and/or a derivate thereof, for example. Protein hydrolysates are product mixtures obtained through the acidically, basically or enzymatically catalyzed decomposition of proteins. As contemplated herein, the expression protein hydrolysates also includes total hydrolysates, as well as individual amino acids and the derivatives thereof, as well as mixtures of various amino acids. The molecular weight of the usable protein hydrolysates is between about 75, the molecular weight for glycine, and about 200,000, preferably from about 75 to about 50,000 and most preferably from about 75 to about 20,000 Daltons.

The cosmetic composition can also contain as a nourishing agent a vitamin, a provitamin, a vitamin precursor and/or a derivative thereof. Such vitamins, provitamins and vitamin precursors are usually assigned to the groups A, B, C, E, F and H.

Further nourishing agents are panthenol, caffeine, nicotinamide and sorbitol.

The cosmetic compositions can also contain, as a nourishing agent, a plant extract, as well as mono- and/or oligosaccharides and/or lipids.

Aerosol dispensing containers are particularly suitable for dispensing and applying the cosmetic compositions. A further subject of the application is the corresponding cosmetic agents, including

a) a cosmetic composition as contemplated herein
b) an aerosol dispensing container.

Aerosol delivery containers are understood to be pressure containers inside which there is a higher gas pressure than outside the container, and from which a gas flow can be removed via a valve. Aerosol dispensing containers, in other words, are pressure containers used to dispense a product (for example, a cosmetic composition) through the internal gas pressure of the container via a valve.

The cosmetic agents can be produced in the normal manner. As a rule, all constituents of the cosmetic composition, with the exception of the propellant, are placed in a suitable pressure-resistant container. This is then closed by employing a valve. The desired amount of the propellant is then added by employing conventional technology.

Vessels made of metal (aluminum, tinplate, tin), protected or splinter-proof plastic or of glass that is coated on the outside with plastic are eligible as pressure-proof containers, with compression strength, tensile strength, corrosion resistance, ease of filling, aesthetic aspects, portability, printability etc. being factors in their selection. Special inner coatings ensure corrosion resistance in relation to the composition packaged in the pressure container. It is particularly preferred that the valves which are used have a valve paint coated on the interior, wherein the coating and valve material are compatible with each other. If aluminum valves are used, their valve heads can be coated on the inside with Micoflex varnish, for example. If tinsheet valves are used, their valve heads can be coated on the inside with PET (polyethylene terephthalate), for example.

A multi-chamber dispenser can also be used as an aerosol dispensing container. The multi-chamber dispenser can be designed in such a way that one chamber is filled with the compressed propellant and another chamber is filled with the remaining constituents of the composition as contemplated herein. One such multi-chamber dispenser is, for example, a so-called bag-in-can package.

The spraying rate of the cosmetic agent is preferably from about 6.5 to about 10.0 g/10 s.

As stated at the outset, the cosmetic compositions as contemplated herein are particularly suitable for cleansing keratinous fibers, more particularly human hair. A corresponding use of said compositions is a further object of this application.

Furthermore, the cosmetic composition is suitable for giving the hair texture and volume. Use of this cosmetic composition for temporary deformation of keratinous fibers, especially human hair, is therefore a further object of this application.

To cleanse keratinous fibers, the cosmetic composition is applied to said fibers. In a further step, the composition can subsequently be removed again from the keratinous fiber at least partially. This can be done, for example, through mechanical effect on the fibers, more particularly by combing or brushing the fibers. Alternatively, the cosmetic composition can be blown out of the hair by employing a hairdryer, for example.

If, after application, the cosmetic agent is removed from the keratinous fibers at least partially, the exposure time of the composition on the fibers is preferably between from about 1 and about 10 minutes.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition comprising, relative to its total weight:
a) from about 1.0 to about 15 wt. % starch;
b) from about 0.05 to about 3.0 wt. % hydrophobized metal oxide powder;
c) from about 0.05 to about 3.0 wt. % silicone oil;
d) from about 2.0 to about 20 wt. % ethanol; and
e) from about 40 to about 95 wt. % propellant.

2. A cosmetic composition according to claim 1, wherein the cosmetic composition also comprises:
f) from about 0 to about 10 wt. % dye(s); and
g) from about 0 to about 10 wt. % pigment(s)
with the proviso that the sum of the quantities of ingredients f) and g) is from about 0.0001 to about 10 wt. %, and is based on the total weight of the cosmetic composition.

3. A cosmetic composition according to claim 1, wherein the weight proportion of starch relative to the total weight of the cosmetic composition is from about 1.5 to about 10 wt. %.

4. A cosmetic composition according to claim 1, wherein the percentage weight of the hydrophobized metal oxide powder to the total weight of the composition is from about 0.07 to about 2.0 wt. %.

5. A cosmetic composition according to claim 1, wherein the percentage weight of the silicon oil to the total weight of the composition is from about 0.07 to about 2.0 wt. %.

6. A cosmetic composition according to claim 1, wherein the percentage by weight of ethanol of the total weight of the cosmetic composition is from about 4.0 to about 15 wt. %.

7. A cosmetic composition according to claim 1, wherein the percentage by weight of propellant of the total weight of the composition is from about 60 to about 92 wt. %.

8. A cosmetic composition according to claim 2, comprising, in terms of their total weight in an amount of from about 0.0001 to about 10 wt. % pigment(s).

9. (canceled)

10. A cosmetic composition according to claim 2, whereby ingredients f) and/or g) are present in the form of a starch-dye compound and/or a starch-pigment compound.

11. A cosmetic composition according to claim 1, wherein the cosmetic composition comprises at least about 90 wt. % of:
a) starch;
b) hydrophobized metal oxide powder;
c) silicone oil;
d) ethanol; and
e) propellant.

12. (canceled)

13. (canceled)

14. A method for cleansing of keratinous fibers wherein the cosmetic composition according to claim 1 is applied to keratinous fibers.

15. A cosmetic composition according to claim 1, wherein the weight proportion of starch relative to the total weight of the cosmetic composition is from about 2.0 to about 5.0 wt. %.

16. A cosmetic composition according to claim 1, wherein the percentage weight of the hydrophobized metal oxide powder to the total weight of the composition is from about 0.1 to about 1.0 wt. %.

17. A cosmetic composition according to claim 1, wherein the percentage weight of the silicon oil to the total weight of the composition is from about 0.1 to about 1.0 wt. %.

18. A cosmetic composition according to claim 1, wherein the percentage by ethanol of the total weight of the cosmetic composition is from about 7.0 to about 12 wt. %.

19. A cosmetic composition according to claim 1, wherein the percentage by weight of propellant of the total weight of the composition is from about 80 to about 90 wt. %.

20. A cosmetic composition according to claim 2, further comprising in terms of their total weight, an amount of from about 0.01 to about 9.5 wt. %, pigment(s).

21. A cosmetic composition according to claim 2, further comprising in terms of their total weight, an amount of from about 0.1 to about 8.5 wt. %, pigment(s).

22. A cosmetic composition according to claim 2, further comprising in terms of their total weight, an amount of from about 0.25 to about 8 wt. %, pigment(s).

23. A cosmetic composition according to claim 1, wherein the cosmetic composition comprises at least about 94 wt. % of the constituents
a) starch;
b) hydrophobized metal oxide powder;
c) silicone oil;
d) ethanol; and
e) propellant.
